(19) United States
(12) Patent Application Publication
(10) Pub. No.: US 2003/0175364 A1
(43) Pub. Date: Sep. 18, 2003

(54) Process for binding carotenoids to proteins and the products thereof

(76) Inventors: Jerry Newman, West Des Moines, IA (US); Fayad Sheabar, West Des Moines, IA (US); Brian Kouba, Walmra, HI (US); Linda A. Fullmer, Ankeny, IA (US); Zoraida DeFreitas, Polk City, IA (US)

Correspondence Address: Kent A. Herink, The Financial Center, Suite 2500, 666 Walnut Street, Des Moines, IA, 50309, US

(21) Appl. No.: 10/086,335

(22) Filed: Mar. 1, 2002

(51) Int. Cl. A61K 35/78

(52) U.S. Cl. 424/725

(57) ABSTRACT

A method for preparing a product of a protein and carotenoid. The carotenoid is added to a solvent. A source of metal ions and a source of proteins is added. The protein and carotenoid product is extracted from the solution in order to demonstrate binding of the lutein to the proteins. The protein and carotenoid product is substantially soluble in water and increases the ability to add the carotenoid to water-based systems, such as foods, beverages, and personal care products. The protein and carotenoid products are particularly suited for use in human supplements where it is desired to add both the protein and the carotenoid to the diet.

### BACKGROUND OF THE INVENTION

1. Field of the Invention

The invention relates generally to a method for binding carotenoids to proteins and, more specifically, to carotenoid-protein compounds which are particularly suited for supplementing foods, beverages, dietary supplements, and personal care products with carotenoids as well as proteins.

2. Background of the Prior Art

Carotenoids are naturally-occurring yellow to red pigments of the terpenoid group that can be found in plants, algae, and bacteria. Carotenoids include hydrocarbons (carotenes) and their oxygenated, alcoholic derivatives (xanthophylls). They include actinioerythrol, astaxanthin, bixin, canthaxanthin, capsanthin, capsorubin, -8′-apo-carotenal (apo-carotenal), -12′-apo-carotenal, -carotene, -carotene, “carotene” (a mixture of - and -carotenes), -carotene, -cryptoxanthin, lutein, lycopene, violerythrin, zeaxanthin, and esters of hydroxyl- or carboxyl-containing members thereof. Many of the carotenoids occur in nature as cis- and trans-isomeric forms, while synthetic compounds are frequently racemic mixtures. The carotenes are commonly extracted from plant materials. For example, lutein extracted from marigold petals is widely used as an ingredient in poultry feed where it adds color to the skin and fat of the poultry and to the eggs produced by the poultry. Many of the carotenes are also made synthetically; much of the commercially available -carotene has been made through synthesis.

Carotenoids are used in the pharmaceutical industry and as ingredients in nutritional supplements, most commonly to date because of their pro-vitamin A activity. They have been extensively studied as antioxidants for protection against cancer and other human and animal diseases. Among the dietary carotenoids, the focus has been on -carotene. More recently, research has begun to elicit the broad role that other carotenoids play in human and animal health. The xanthophylls in particular have been shown to possess strong antioxidant capabilities and may be useful in reducing the risk of disease. For example, the consumption of lutein and zeaxanthin has been identified as leading to a 57 percent reduction in age-related macular degeneration (Seddon et al., 1994. J. Amer. Med. Assoc. 272(18): 1413-1420). Lycopene has been identified as a nutrient that is active in reducing the risk of prostate cancer.

Carotenoids have also been of wide interest as a source of added color for food and drink products and many efforts have been made to attempt to use them as “natural” colorants for foods and beverages. However, their insolubility in water, their low solubility in fats and oils, high melting points, and their sensitivity to oxidation has limited their use, particularly in water-based products such as beverages and juices and products to which water is to be added.

Proteins are, of course, widely found in foods and beverages. Moreover, it has been common for many years to supplement foods and beverages with proteins to improve their nutritional value, affect taste or other sensory characteristics, change their physical properties, and the like. Proteins are also widely found in and added to personal care products for many of the same reasons. The largest segment of proteins added to many products is derived from soybeans. The nutritional and health benefits of soy proteins are primary factors that are affecting the use and growth in use of soy proteins. While the nutritional benefits of soy proteins have been widely recognized since at least the 1970's, only in the last decade has the health benefits come to be widely recognized. Soy proteins have become popular as additions to nutriceutical products such as nutrition bars, sports beverages, and infant formula. Many proteins, including soy proteins, have a number of functional characteristics that make them attractive for use in foods, beverages and personal care products. These attributes include solubility, emulsification, fat absorption, hydration, and texture enhancement.

An attempt has been made to utilize a combination of proteins and carotenoids. A protein isolate derived from pea pods was applied as a matrix material for the microencapsulation of -carotene, intended for personal care applications. Harmsen, P. F. H., et al. Microencapsulation of -carotene by supercritical CO₂ technology. IFSCC Magazine. 4, 34-36 (2001). Microspheres of the pea protein were prepared and combined in a supercritical CO₂ reactor with -carotene. The -carotene was absorbed into the porous matrix of the microspheres. There was no reaction between the pea protein and the -carotene.

### SUMMARY OF THE INVENTION

The invention consists of a method for creating compounds of proteins and carotenoids, which for the purposes of this description are either carotenoids or esters of the carotenoids. The carotenoid is added to a protein and a source of metal ions and reacted under basic conditions at a temperature of between about 0° C. and about 50° C. for a time between about 30 minutes and about 72 hours. Suitable proteins include soybean proteins and isolates, such as soy milk powders and drinks; albumin from various sources (i.e., bovine and chicken); -globulins; soybean Kunitz and Bowman-Birk proteinase inhibitors; potato proteinase inhibitors, carboxypeptidase inhibitor and others; whey proteins, concentrates and isolates; isoflavone containing protein product; corn germ proteins; oat flower; and hydrolyzed vegetable proteins. The protein may either be specific protein products or may be proteins found in the food, beverage, or personal care product being supplemented. Suitable carotenoids include actinioerythrol, astaxanthin, bixin, canthaxanthin, capsanthin, capsorubin, -8′-apo-carotenal (apo-carotenal), -12′-apo-carotenal, -carotene, -carotene, “carotene” (a mixture of - and -carotenes), -carotene, -cryptoxanthin, lutein, lycopene, violerythrin, zeaxanthin, and esters of hydroxyl- or carboxyl-containing members thereof, although the oxygenated xanthophylls and their esters are preferred. Particularly suited are lutein and zeaxanthin and their esterified forms. Suitable metal ions include Mg⁺⁺, Mn⁺⁺, Fe⁺⁺⁺, K⁺, and Ca⁺⁺. It may be desired to first dissolve the carotenoid in a solvent. Suitable solvents include tetrahydrofuran, methanol, isopropanol, ethanol, propylene glycol, and methylene chloride.

Products of this process may be used in topically applied personal care products, various food and beverage products and dietary supplements and could serve as a delivery system for the subject carotenoid and protein in various food products, beverages and supplements as well as topically applied personal care and health products. Alternatively, the products of the present invention may be formed in the food, beverage, dietary supplements, or personal care product itself during a stage or stages in the manufacturing process of the food, beverage, or personal care product. In addition, the present invention provides a product that includes proteins that can be selected to have positive functional properties in humans and animals. Others did not attempt achieving these goals. The binding of a carotenoid to various proteins and at various ratios under the tested conditions was not expected.

### BRIEF DESCRIPTION OF THE FIGURES

FIG. 1 is a representative HPLC chromatograph of a soybean protein-carotenoid compound formed using the method of this invention.

### DETAILED DESCRIPTION OF THE PREFERRED EMBODIMENTS

The carotenoid used in the method may be of any desired form that permits the carotenoid to go into solution. Preferably, the carotenoid will be freshly prepared or in a protected form so that it has a high activity. In particular, use of the carotenoid ina pure form or in the form of powder (sometimes referred to as “dry cake”), beadlets or microspheres is convenient. In the preferred embodiments described herein, the carotenoids were prepared fresh on a daily basis. In certain of the examples, the carotenoid was prepared via the method described in the section on preparing reagent grade carotenoid and further purified using the method described in the section on the preparation of analytical grade carotenoids

The proteins to be used in the method may also be of any suitable or desired form that will go into basic aqueous solution for reaction with the carotenoids. The proteins are preferably dissolved in a basic buffer, such as an aqueous solution of Tris, carbonate salts, glycine, or the like, having pH >8.5. Preferred proteins are soybean proteins and isolates; albumin from various sources (i.e., bovine and chicken); -globulins; soybean Kunitz and Bowman-Birk proteinase inhibitors; potato proteinase inhibitors; whey proteins, concentrates and isolates; isoflavone containing protein products; corn germ proteins; oat flour; and hydrolyzed vegetable proteins.

Alternatively, the proteins may be present in a food, beverage, dietary supplements, or personal care product. For example, if it was desired to supplement a soy milk drink with a carotenoid, the carotenoid source is added to the soy milk drink prior to the heat processing step. If the soy milk drink has been supplemented with a metal, such as iron or calcium (as is very often the case), the products of the present invention will form during the heat processing step.

A source of metal ions is added to the reaction solution if they are not already present. Preferred metal ions include Mg⁺⁺, Mn⁺⁺, Fe⁺⁺⁺, K⁺, and Ca⁺⁺.

The reactions may be carried out in a suitable solvent, such as ethanol, methanol, tetrahydrofuran, isopropanol, propylene glycol, and methylene chloride.

### EXAMPLE 1

Preparation of Reagent Grade Lutein

The purpose of this preparation is to purify lutein from the source lutein dry cake (Kemin Foods, L.C.) and for use of the purified lutein in a subsequent preparation of analytical grade lutein using column chromatography. The materials used are fresh lutein dry cake, distilled water, HPLC grade reagent alcohol (Fisher), HPLC grade hexane (Fisher), gas chromatography resolve methylene chloride (Fisher), and argon gas. Six grams of lutein dry cake is suspended in 100 ml of cold water (<10° C.) and stirred until a slurry is created. The slurry is filtered using Whatman #4 filter paper, washed with 50 ml cold water (<10° C.), washed three times with 50 ml cold ethanol (<10° C.), and then washed three times with cold hexane (<10° C.).

The crystals are dissolved in a minimal amount of room temperature methylene chloride. If the crystals have not dissolved after 200 ml, the crystals are filtered again and any solid on the filter paper is discarded. Hexane is added to the dry cake/methylene chloride solution until the solution becomes turbid, typically between about 600 ml and about 800 ml hexane. The solution is stored undisturbed at less than 10° C. for at least 8 hours. The crystals are collected on a fine glass frit and washed with 25 ml of cold hexane (<10° C.). The crystals are then dried by pulling vacuum on the filter while blanketing the crystals under argon. The crystals are stored in an amber vial at less than −10° C.

### EXAMPLE 2

Preparation of Analytical Grade Lutein

The purpose of this preparation is to purify lutein obtained from the preparation of Example 1 by column chromatography. The materials used are HyfloSuper Cel (Aldrich or Fisher), adsorptive magnesia (Fisher), sea sand (Fisher), HPLC grade acetone (Fisher), HPLC grade hexane (Fisher), methylene chloride, and argon gas. Equal parts of adsorptive magnesia and HyfloSuper Cel are used as the adsorbent. The column is dry-packed with one-fourth inch of sea sand, 8 in of adsorbent, and another one-fourth inch of sea sand. For a column with an inside diameter of 2 inches, 150 g of adsorbent are used. Using an analytical balance, 180 mg of reagent grade lutein is weighed and dissolved in 50 ml of acetone. The solution is filtered through a 2 μm filter. Using a transfer pipette, the solution is added to the column and blown dry with warm air, approximately one to one and one-half hours. When the column is dry, 500 ml of a 19:1 hexane/acetone mixture is added and the column is eluted until clear. When the solvent level is near the top of the adsorbent in the column, a 13:7 mixture of hexane/acetone is added. The first light orange band, which is lutein, is collected. The second reddish orange band, which is zeaxanthin, is also collected. A rotary evaporator is used to remove the solvent from the lutein-containing fractions. All fractions are combined and a minimal amount of methylene chloride (<10 ml) is added. Hexane is added until the solution becomes cloudy or the volume reached 300 ml. The mixture is placed in a freezer for at least 8 hours to allow for recrystallization. The solution is filtered under reduced pressure on a fine glass frit filter and dried under reduced pressure using a blanket of inert gas, such as argon. The lutein is transferred into a pre-weighed amber flask, covered with a Kimwipe® (Kimberly-Clark) and filtered under reduced pressure in a desecrator for 10-16 hours. The cap of the flask is wrapped with Parafilm® (Pechiney Plastic Packaging) and the lutein crystals are stored in the freezer until used.

### EXAMPLE 3

Binding of Lutein to Selected Proteins

The carotenoid used in this Example 3 was lutein in dry cake form (Kemin Foods, L.C., Des Moines, Iowa). The cake was dissolved in THF at a concentration of approximately 5.28 mM (3 mg lutein in 1 ml THF). Four hundred nanograms of a selected protein (bovine and chicken albumin, gamma-globulins, Kunitz inhibitor, Bowman-Birk inhibitor, whey, and soy protein isolates 873 and 891 from Archer Daniels Midland, DeKalb, Ill.) were added to 200 μl Tris buffer (1.74 g Trizma crystals and 0.42 g calcium chloride added to 250 ml de-ionized H₂O, pH 8.74) together with 2 μl of the lutein/THF solution. These reaction solutions were prepared in microcentrifuge tubes (1.5 ml), mixed, and allowed to react at 4° C., overnight, and in the dark. In addition, all reactions were performed in the presence of an alternative metal ion selected from the group Mg⁺⁺, Mn⁺⁺, Fe⁺⁺⁺, K⁺, and Ca⁺⁺(MgCl₂ 6H₂O, MnCl₂4H₂O, FeCl₃ 6H₂O, KCl, and CaCl₂) at 60 μM concentration.

In order to demonstrate the binding of lutein to proteins under the developed conditions, extractions were conducted using a hexane wash of 200 μl which was added to the microcentrifuge tubes which were then vortexed and subsequently put in a centrifuge for approximately 15 s. The hexane wash, vortex and centrifuge procedure was repeated six times. The supernatant was extracted and combined from all six washes and used later to compare absorbance vs. aqueous phase.

Protein and lutein content in the protein solutions and solvents used for extraction were determined by measuring the absorbance at 280 and 446 nm using a spectrophotometer. These data were then compared to determine effectiveness of individual reactions between the selected carotenoid and the selected protein.

The results are compiled and are reported in weight:weight or mol:mol (when available) ratios.

Binding of Lutein to Proteins

| | Lutein/Protein Ratio | | | | | | | | | |
|---|---|---|---|---|---|---|---|---|---|---|
| | w/o metal | | | | | | | | | |
| | w/o metal ions | | Fe³⁺ | | Ca²⁺ | | K⁺ | | Mg⁺ | |
| Protein | w/w | mol/mol | w/w | mol/mol | w/w | mol/mol | w/w | mol/mol | w/w | mol/mol |
| Bowman-Birk | 4.69 | 66 | 6.68 | 94 | 3.91 | 55 | 5.06 | 71 | 6.64 | 94 |
| Bovine Serum | 2.59 | 306 | 3.1 | 365 | 4.29 | 506 | 3.24 | 383 | 2.49 | 294 |
| Albumin | | | | | | | | | | |
| Chicken Serum | 1.33 | 105 | 0.9 | 71 | 0.84 | 67 | 1.2 | 95 | 1.16 | 116? |
| Albumin | | | | | | | | | | |
| Kunitz | 1.17 | 46 | 0.74 | 29 | 0.78 | 31 | 0.6 | 24 | 0.74 | 29 |
| Gamma Globulin | 0.67 | | 1.08 | | 2.4 | | 1.26 | | 2.34 | |
| Soy Protein 873 | 0.28 | | 0.08 | | 0.11 | | 0.41 | | 0.69 | |
| Whey Proteins | 0.05 | | 0.22 | | 0 | | 0.05 | | 0.22 | |
| Soy Proteins 891 | 0.28 | | 0.08 | | 0.11 | | 0.41 | | 0.69 | |

### EXAMPLE 4

High Concentration Experiment of Lutein and Soybean Protein Isoflavones

The study described in Example 3 used ng levels of protein-isoflavone product and indicated that incubating a protein solution with lutein brought about a subsequent increase in the absorbance at 446 nm, which our tests determine are the result of an increase in the amount of lutein in the solution, even following several extractions with hexane and mythelene chloride. This increase was not observed in a control sample (i.e., protein solution incubated without the addition of lutein.

Two hundred mg of isoflavones (ADM) were weighed out and placed in a 20 ml vial to which 20 ml of Tris-HCl buffer (pH ˜8.3: 1.74 g Trizma crystals, 0.42 g calcium chloride, and 250 ml water) was added. Four mg of lutein was dissolved in 1 ml THF in a 1.5 ml microcentrifuge tube and then vortexed to mix thoroughly.

From the lutein solution, 200 μl was drawn out and added to the protein solution—the entire mixture was vortexed to ensure complete mixing and allowed to sit for three days at room temperature while covered in aluminum foil.

A second reaction was also prepared, except that lutein addition was omitted from the process for control purposes. At the end of the incubation period, the lutein/protein was allowed to settle to the bottom of each vial. The vials containing the supernatant were extracted with 10 ml hexane and allowed to separate. After complete mixing, an aliquot of 2.5 ml was taken and placed in a 10 ml test tube (for both protein (+lutein) and protein (−lutein) liquids). The two aliquots were centrifuged and the hexane layer was aspirated and placed in a separate vial. Three additional extractions were performed on each sample using 2.5 ml hexane. Absorbance at 446, 460, and 280 nm in the combined hexane extracts was performed and no difference was noted between the protein (+lutein) and protein (−lutein) preparations.

The protein sediment collected at the bottom of the original 20 ml vials was washed with 20 ml methylene chloride (which was saved and transferred for future analysis).

After washing, the solids were dried under reduced pressure (in 10 ml test tubes) for approximately 45 minutes and then placed in a desiccator under reduced pressure overnight.

The next day, each of the solids samples were reconstituted using 10 ml of Tris-HCl buffer in their respective tubes. From each, 2.5 ml aliquots were withdrawn and placed into new 10 ml test tubes for extraction using methylene chloride.

Overall, each sample was extracted 6 times with 2.5 ml methylene chloride in order to remove all unbound lutein. All methylene chloride extracts were combined with the original 20 ml hexane extracts. Protein solids were dried under reduced pressure for ˜1 hour and then desiccated under reduced pressure overnight. Four ml of acetonitrile were added to each 10 ml tube and heated for 30 minutes (60° C.), followed by repeated sonication/vortexing until fully dissolved and additional 16 ml of acetonitrile were added. Samples were allowed to settle and analyzed at 280, 446, 460, and 480 nm.

After UV analysis, the protein solids were split into two portions and dried under reduced pressure for approximately five hours and placed in the desecrator under reduced pressure overnight. Solids were reconstituted using Tris-HCl buffer to a final volume of 20 ml buffer. After thorough mixing, absorbance at 380, 446 and 460 nm was performed. In addition, HPLC analysis was performed on the protein samples using a C4 Vidac Reversed Phase column (MetaChem Technologies, Inc.) with a 95:5 water to acetonitrile mobile phase ratio (three vials including protein+lutein, protein−lutein, and lutein standard).

Absorbance at various wavelengths (i.e., 280, 446, 480, and 260 nm) of the acetonitrile reconstituted protein solids previously extracted with methylene chloride to remove any unbound lutein (1-20 ml wash along with 6-2.5 ml washes). Results shown in the following tables indicate that both protein (−lutein) and protein (−lutein) had absorbance at 280 and 460 nm. However, the protein (+lutein) solution showed a larger absorbance at 446, 460, and 480 nm in comparison to protein (−lutein) indicating that even after all the extensive extraction, lutein was still attached to the protein.

Absorbance at Various Wavelengths
of Purified Lutein-Protein Samples

| | 280 nm | 446 nm | 460 nm | 480 nm | 260 peak |
|---|---|---|---|---|---|
| Protein | 2.97455 | 1.5427 | 1.5354 | 1.5144 | 3.50386 |
| (−Lutein) | | | | | |
| Protein | 2.9817 | 1.75535 | 1.77965 | 1.7555 | 3.414085 |
| (+Lutein) | | | | | |
| Shift | 0.00715 | 0.21265 | 0.24425 | 0.2411 | −0.089775 |

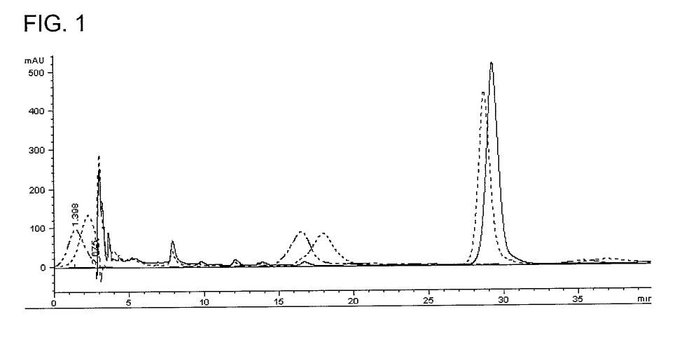

HPLC chromatographs (FIG. 1) showed slight shifts in retention time following the reaction indicating the formation of new products during the reaction.

The experiment was repeated and the reaction was allowed to proceed over a three-day period. Reaction was also performed in the presence of CHAPS (3-[(3-cholamidopropyl)dimethylammonio]-1-propanesulfonate) that acts as a surfactant to see if it may have any positive effect on protein/lutein binding. A procedure similar to the one described in this example was followed with the exceptions that no hexane extractions were performed and proteins were constituted in buffer. Protein solids were extracted with methylene chloride and the protein solution was dried down and then combined with the remaining protein solids that were separately extracted by methylene chloride. After solvent removal and reconstitution in 20 ml of Tris-HCl buffer, a protein sample was diluted 1:9 (0.5 ml solution:4.5 ml buffer) and absorbance determined at various wavelengths.

| | 280 nm | 446 nm | 460 nm | 480 nm |
|---|---|---|---|---|
| Protein + Lutein − | 1.3632 | 0.27393 | 0.26903 | 0.26502 |
| CHAPS | | | | |
| Protein + Lutein + | 1.150.3 | 0.17777 | 0.17619 | 0.17309 |
| CHAPS | | | | |
| Protein + | 0.74875 | 0.12549 | 0.12336 | 0.12213 |
| CHAPS | | | | |
| Protein − | 0.80708 | 0.15947 | 0.15757 | 0.15444 |
| CHAPS | | | | |

Once again, the incubation of lutein along with protein brought about an increase in the absorbance at all wavelengths. CHAPS had a beneficial effect on the binding efficiency as evidenced by the increase in absorption at 446 nm of the sample containing lutein. These results support the previous findings that incubating lutein with protein brings about an increase in its content in the precipitate and not the liquid.

Based on the absorbance results determined in the various samples and based on the extension coefficient levels of lutein were calculated and used to determine the lutein:protein binding ratio.

| | 280 nm | 446 nm | 460 nm | 480 nm |
|---|---|---|---|---|
| Protein − | 0.80708 | 0.15947 | 0.15757 | 0.15444 |
| CHAPS | | | | |
| Protein + Lutein − | 1.3632 | 0.27393 | 0.26903 | 0.26502 |
| CHAPS | | | | |
| Difference | | 0.11446 | | |

Finally, after using factors to convert these concentrations back to an original amount of lutein complexed in the protein solids recovered, one finds the following:

| | Lutein Added to | Lutein Found | Percentage of |
|---|---|---|---|
| | Reaction | Complexed | Lutein Reacted |
| First Trial | 0.8 mg/reaction | 0.03156 mg | 3.95% |
| Second Trial | 0.8 mg/reaction | 0.3695 mg | 46.19% |

Binding results observed in the second large-scale trial were about 10 times higher than the first experiment (0.158, 1.845 w/w lutein/protein ratio, in the 1^{st} and 2^{nd} trials, respectively). This difference could be attributed to the fact that no mixing was applied during incubation in the first experiment. In addition, losses during the extraction process could have contributed to the differences also. However, it should be noted that the binding results of the second binding experiment performed at the high scale (1.845 w/w lutein/protein) were very similar to the results of the binding experiment performed with the same protein, however, at a ng level scale (1.77 w/w lutein/protein).

Summary of Lutein-Protein Binding Results
7/26 Lutein/Protein Binding

| Protein/Treatment | ng/ng Ratio | | mol/mol Ratio |
|---|---|---|---|
| Bovine Serum | 2.59, 3.69, 2.41 | +/−0.69 | 305.63, 435.27, 284.17 |
| Albumin | | | |
| w/o metal | | | |
| Ferrous | 3.09, 4.22, 3.59 | +/−0.56 | 365.33, 497.54, 422.76 |
| Calcium | 4.29, 3.25, 3.63 | +/−0.53 | 505.37, 382.61, 427.85 |
| Potassium | 3.24, 2.33, 3.00 | +/−0.48 | 382.55, 274.18, 353.27 |
| Magnesium | 2.49, 3.41, 3.59 | +/−0.59 | 294.09, 402.50, 423.55 |
| Chicken Serum | 1.33, 0.26 | +/−0.76 | 105.24, 20.21 |
| Albumin | | | |
| w/o metal | | | |
| Ferrous | 0.90, 0.71 | +/−0.14 | 71.66, 56.34 |
| Calcium | 0.84, 0.35 | +/−0.35 | 66.22, 27.45 |
| Potassium | 1.19, 0.37 | +/−0.58 | 94.85, 29.57 |
| Magnesium | 1.16, 0.88 | +/−0.20 | 92.06, 69.81 |

Kunitz Inhibitor (ng/ng)

| | |
|---|---|
| w/out metal | 1.17 |
| Ferrous | 0.74 |
| Calcium | 0.78 |
| Potassium | 0.60 |
| Magnesium | 0.74 |

Bowman-Birk Inhibitor (ng/ng)

| | |
|---|---|
| w/o metal | 4.69 |
| Ferrous | 6.68 |
| Calcium | 3.91 |
| Potassium | 5.06 |
| Magnesium | 6.64 |

Gamma-Globulins (ng/ng)

| | |
|---|---|
| w/o metal | 0.67 |
| Ferrous | 1.08 |
| Calcium | 2.40 |
| Potassium | 1.26 |
| Magnesium | 2.34 |

Whey Proteins (ng/ng)

| | |
|---|---|
| w/o metal | 0.05 |
| Ferrous | 0.22 |
| Calcium | −0.01 |
| Potassium | 0.05 |
| Magnesium | 0.22 |

Soy Isolate 873 (ng/ng)

| | | |
|---|---|---|
| w/o metal | 0.28, 0.69 | +/−0.29 |
| Ferrous | 0.08, 0.67 | +/−0.42 |
| Calcium | 0.11, 0.78 | +/−0.47 |
| Potassium | 0.41, 0.48 | +/−0.05 |
| Magnesium | 0.69, 0.41 | +/−0.20 |

Soy Isolate 891 (ng/ng)

| | |
|---|---|
| w/o metal | 0.0 |
| Ferrous | 0.09 |
| Calcium | 0.13 |
| Potassium | 0.01 |
| Magnesium | 0.15 |

Isoflavone Compound (ng/ng)

| | |
|---|---|
| w/o metal | 1.77 |
| Ferrous | 1.74 |
| Calcium | 1.56 |
| Potassium | 1.99 |
| Magnesium | 1.97 |

Oat Flour (ng/ng)

| | |
|---|---|
| w/o metal | 1.53 |
| Ferrous | 1.70 |
| Calcium | 1.42 |
| Potassium | 1.41 |
| Magnesium | 1.56 |

Corn Germ (ng/ng)

| | |
|---|---|
| w/o metal | 1.77 |
| Ferrous | 1.61 |
| Calcium | 1.49 |
| Potassium | 1.54 |
| Magnesium | 1.54 |

Whey Concentrate (ng/ng)

| | |
|---|---|
| w/o metal | 2.33 |
| Ferrous | 1.83 |
| Calcium | 1.30 |
| Potassium | 1.77 |
| Magnesium | 1.57 |

### EXAMPLE 5

Binding of Lutein to Protein Using Soy Isoflavones in Two Different Solvents and at a Low Lutein:Protein Ratio

Sixteen hundred mg of Soy Isoflavones (ADM, lot#0011101) were dissolved in 40 ml Tris buffer by stirring. Seven aliquots of 5 ml of this stock solution were placed in 20 ml vials, 200 mg in each vessel. As in Example 4, to each of these 15 ml of Tris buffer was added. New stock lutein solutions were made in ethanol and propylene glycol. The lutein stock used was Dry Cake Lutein lot# 082346-04. To each of three 2 ml centrifuge tubes, 4 mg of the dry cake were added. To each of the samples, a lutein solution, prepared in different solvents, was added, whereas only solvents (250 μl) were added to their respective controls. The sample to which lutein/THF was added was used as a positive control. In order to test the efficiency of the reaction without solvent, 1 mg of dry cake was added to 200 mg protein dissolved in 15 ml Tris buffer. In this case, the corresponding control consisted of 200 mg protein dissolved in 15 ml Tris buffer.

In order to test the efficiency of the reaction with a more diluted lutein solution, an experiment was set in which a 5 times more diluted lutein solution than originally used was prepared, consisting of 3 mg lutein in 3.75 ml each ethanol and propylene glycol. Eight hundred mg of Soy Isoflavones were weighed out. Five ml of samples of the soy isoflavone solution were aliquoted into each of four new 20 ml vials, along with 15 ml of Tris buffer. To each of the test vials, 1.25 ml of the diluted lutein solutions were added, whereas 1.25 ml of the solvents were added to the corresponding controls, respectively.

Another set of samples was set in order to test the efficiency of the reaction at a ratio that is 2.5 times smaller than the one originally used (i.e., 0.4 mg lutein/200 mg proteins instead of 1 mg lutein/200 mg protein). For this purpose, 0.4 mg lutein in 250 μl THF or 250 μl THF were added to soy isoflavone solutions (200 mg soy isoflavone in 20 ml Tris buffer), respectively.

Observations at this point revealed the following results:

Highest Color Intensity Least Color Intensity

Dil. EtOH [symbol] Positive Control [symbol] Undil. EtOH [symbol] Lut/THF [symbol] Dil. PG [symbol] Undil. PG [symbol] Dry Cake

All vials were allowed to stir for twenty-four hours and then placed in the freezer.

The vials were thawed and transferred to 50 ml conical tubes. Tubes were vortexed at 4000 rpm for 5 min, and the supernatant was aspirated. Twenty ml of methylene chloride were added. The vials were vortexed and centrifuged at 4000 rpm for 5 minutes. Methylene chloride was aspirated. The residues of the solvent were removed using an argon stream. A second wash with methylene chloride was performed. Solvent was aspirated from the tubes, and they were placed in the 4° C. refrigerator until the next day, whereupon the solvent residues were removed with a stream of argon gas. The samples were fully dried using a lyophillizer for 4 hours and placed in the 4° C. refrigerator until further analysis was performed.

### EXAMPLE 6

Binding of Lutein to Proteins Using Various Solvents at a Low Ratio and Using an Edible Buffer

Four mg of dry cake lutein (lot#082346-04 stored at −20° C. in a freezer) was weighed into two 7 ml glass vials and 5 ml of ethanol or isopropanol were added.

To prepare the control and test samples, 1600 mg of Isoflavone Soy Product (ADM lot#0011101) were weighed into a beaker and brought up to 40 ml with Tris buffer (1.74 g Trizma crystals, 0.42 g CaCl2, and 250 ml de-ionized water). Five ml of the solution was aliquoted into four 20 ml glass scintillation vials. To these four vials, 15 ml of Tris buffer was added. To the controls, 1 ml ethanol or isopropanol were added, whereas 0.5 ml lutein solution in the corresponding solvent was added to the samples, respectively. A sample to which lutein/THF (1 mg/1 ml) was added served as the positive control. The selected ratio and concentrations applied in this experiment are based on the results in Example 5 that indicated that the binding reaction is efficient under those conditions as well.

In order to test the efficiency of the binding reaction in an edible buffer, a sodium carbonate-sodium bicarbonate buffer was prepared. Reagents used were sodium carbonate monohydrate (124.01 g/L) and sodium bicarbonate (84.01 g/L). A 0.1 M solution of each was made and they were mixed at a 1:10 ratio (1 part of sodium carbonate solution added to 9 parts sodium bicarbonate solution). Two hundred mg of the Isoflavone product were weighed into two new 20 ml vials and 20 ml of the sodium carbonate-sodium bicarbonate buffer were added. To control and test samples, 0.5 ml of ethanol and 0.5 ml of the lutein/ethanol solution were added, respectively.

In order to demonstrate the efficiency of the binding reaction with various lots of soy isoflavone, a second lot of ADM Isoflavone Soy Product (lot#000121) was used. Two hundred mg of this compound were weighed into two 20 ml vials. Fifteen ml of Tris buffer were added to each one. To the control and test samples, 0.5 ml of ethanol and lutein/ethanol solution were added, respectively.

All these vials were placed on a stir plate and allowed to stir overnight.

Highest Color Intensity Least Color Intensity

Isoflav+Isoprop/Lutein [symbol] Isoflav Pos. Control [symbol] 2^{nd} lot of Isoflav [symbol] Isoflav w/Bicarb buffer

### EXAMPLE 7

Binding of Lutein to Bovine Serum Albumin, Gamma Globulin, Chem West Soy Protein Powder, and Solgen 40 Extract

This Example 7 was directed at performing binding of lutein to soy proteins of various sources and to other types of proteins (i.e., albumin and -globulin) at a mg scale.

Eight hundred mg of four proteins (Solgen 40 Extract from Solbar Plant Extracts (no lot# apparent), Bovine Serum Albumin (lot#78H0696), Chem West Soy Protein Powder (lot#141193), and Gamma Globulin Bovine (lot#40K7602)) were dissolved in 20 ml Tris buffer (1.74 g Trizima crystals, 0.42 g calcium chloride, and 250 ml de-ionized water, pH 8.7). Aliquots of 5 ml (200 mg protein) of the protein stock solution were transferred into two new 20 ml vials. Fifteen ml of Tris buffer were added to each vial and the control vessel received 250 μl of THF, while the test vessel got 250 μl of Dry Cake Lutein/THF solution (stock was 4 mg Dry Cake Lutein lot# 082346-04/1 ml THF freshly prepared). The solutions were mixed at ambient temperature over a three-day period. Following incubation, 10 ml of the supernatant were taken from all vials and transferred to new 20 ml vials, while the precipitate and excess supernatant were left in the original vial and stored at 4° C. At times when samples were not worked with, they were kept at 4° C.

Ten ml HPLC grade hexane were added to each supernatant tube and samples were centrifuged at 4000 rpm for 5 min. Then the hexane layer was aspirated. This was repeated until there was no color evident in the hexane layer. After washing, all supernatant samples were placed in the analytical evaporator for 6 hours to remove solvent residues. The sample volume was brought to 10 ml using Tris buffer and centrifuged to separate undissolved matter. Then, several dilutions were made depending on the turbidity of the sample, and all absorbance at 280 nm and 446 nm was recorded.

Twenty ml of methylene chloride were added to all protein precipitate samples. Vials were shaken, and methylene chloride layer was aspirated off the bottom. Solvent residues were removed either under reduced pressure or with a stream of argon. All vials were brought to 20 ml using Tris buffer. Appropriate dilutions were made and absorbance at 280 nm and 446 nm was determined in each sample.

The results are as follows:

| | Est. Sup + Est. Ppt. | Est. Sup + Est. Ppt. |
|---|---|---|
| Sup + Ppt. | mg Lutein/200 mg solids | mg Lutein/1 g solids |
| Solgen | 0.302411359 | 1.512056796 |
| CWSoy | 0.446774365 | 2.233871823 |
| Gamma Globulin | 0.379701724 | 1.898508619 |
| BSA | 0.075185 or 0.039837 | 0.199185 |

### EXAMPLE 8

Gram Scale Binding of Protein (Soy Isoflavone, Solgen 40 Soy Protein Extract, Pro Fam 892 Soy Protein Isolate, and Chem West Soy Protein Powder) and Lutein

Two grams of protein (Soy Isoflavone (ADM lot#0011101), Solgen 40 Soy Protein Extract (Solbar no lot#), Pro Fam 892 Soy Protein Isolate (ADM lot#01032261), and Chem West Soy Protein Powder (lot#141193)) were dissolved in 200 ml Tris buffer. Two and one-half ml of THF and lutein cake solution (80mg of lutein dry cake lot#082346-04 dissolved in 20 ml THF) were added to the control and test samples, respectively. The solutions were allowed to stir and interact overnight.

After incubation, samples were transferred to 500 ml separatory funnels. Two hundred ml HPLC grade hexane were added to each funnel and mixed for approximately a minute. The aqueous layer was reacted with methylene chloride (200 ml). Water or centrifugation (4000 rpm for 5 minutes) were used to break the emulsions formed in the protein/lutein samples. Solvents were removed under reduced pressure at 50° C. using a RotoVap. Precipitates of all samples were frozen at −80° C., and lyophillized for approximately three days. The samples were placed in the 4° C. refrigerator until further analysis.

### EXAMPLE 9

Binding of Lutein to Soy Isoflavones and Using Various Buffers and pH Values

Eight samples of Soy Isoflavone Product (ADM lot#0011101) (200 mg) were weighed. To the positive control, 20 ml of the Tris buffer (1.74 g Trizima crystals, 0.42 g calcium chloride, 250 ml de-ionized water; pH 8.7) were added. A new Tris buffer in which the calcium chloride was omitted (the buffer then only contained 1.74 g Trizma pre-set crystals and 250 ml de-ionized water) was made. Twenty ml of this buffer were added to each of 2 new protein samples. The pH of the different Tris buffer sample without calcium chloride was increased to 11.1 using 1.0 M of NaOH. In addition, the efficiency of the binding reaction using a carbonate-bicarbonate buffer was also tested. In this experiment, a 100 ml aliquot of the carbonate buffer (previously described in Example 6) was adjusted to pH 8.2 using 1.0 M HCl. Twenty ml of this buffer were added to each of the 2 new protein samples. Finally, 4 mg of dry cake lutein (lot#082346-04) was weighed out and 5 ml of ethanol was added. This was vortexed and 0.5 ml of the solution was added to each “test” sample. Each control sample received 0.5 ml of ethanol. All vials were placed on stir plates and allowed to stir for two days.

### EXAMPLE 10

Binding of Lutein to Isoflavones, Pro Fam 892 Isolated Soy Protein, 90% Whey Protein Isolate, and Whole Oat Flour

Eight hundred mg of four proteins (ADM Soy Isoflavone Product (lot# 0011101), Pro Fam 892 Isolated Soy Protein (lot# 01032261), Hoogwegt 90% Whey Protein Isolate (lot# OS309), and ConAgra Whole Oat Flour (no lot# apparent)) were dissolved in 20 ml Tris buffer (1.74 g Trizma crystals, 0.42 g calcium chloride, and 250 ml de-ionized water, 8.7 pH). Two aliquots of 5 ml of each of the stock protein solutions (200 mg) were transferred into two new 20 ml vials, and 15 ml of Tris buffer were added to each sample. Two hundred and fifty μl of THF, and 250 μl of Dry Cake Lutein/THF solution (stock was 4 mg Dry Cake Lutein lot# 082346-04/1 ml THF, freshly prepared) were then added to the control and test samples, respectively. This gave four control, and four test vessels. The solutions were mixed overnight. Ten ml of the supernatant were transferred to polypropylene conical tubes, while the precipitate and excess supernatant was left in the original vial and stored at 4° C. When not in use, the samples were stored at 4° C.

Ten ml HPLC grade hexane was added to each supernatant tube and the tubes were centrifuged at 4000 rpm for 5 min. Then the hexane layer was aspirated. Extraction with hexane was repeated until there was no color evident in the hexane layer. Samples were placed in the analytical evaporator for 3 hours to get rid of excess hexane. Protein samples were dissolved in Tris-HCl buffer, dilutions were made depending on clarity of sample, and absorbance at 280 nm and 446 nm was determined.

Twenty ml of methylene chloride was added to all precipitate proteins, samples were mixed, and the methylene chloride layer was aspirated. Samples were left in the fume hood to remove methylene chloride residue. Volumes of all samples were brought to 20 ml using Tris buffer. Appropriate dilutions were made and absorbance at 280 nm and 446 nm was determined.

The results are as follows:

| Sup + Precipt | mg Lutein/1 g solids |
|---|---|
| WPI | 0.2475035 |
| Oat | 0.8360585 |
| ISO | 1.895425415 |
| S892 | 1.981527625 |

Since some of the material was not recovered, the solid weight and protein content left in all the supernatants and precipitate test vessels were of interest. The first process to determine the weight of the solids in the samples started with placing 1 ml of sample into a pre-weighed 5 ml test tube, then weighing again to obtain the sample weight before drying. All samples were vortexed prior to aliquoting into the tube. These samples were placed in the speed vacuum for approximately eight hours until the entire liquid portion had evaporated. Then they were weighed again after drying to obtain the dry sample weight. The amount of solids started with was 200 mg of protein in each control and test vessel, but samples also contained 172.8 mg buffer solids, and 1.0 mg lutein solids. This gave an expected total of 373.8 mg solids. For each protein, the controls for both matrixes were combined, i.e. Oat supernatant control dry wt.+Oat precipitate control dry wt., and Oat supernatant sample dry wt.+Oat precipitate sample dry wt. Then these combined weights were subtracted from the initial weight of the protein and solids, this difference was divided by the initial weight to get the percentage lost. This was done for every set of proteins.

From the first part of the experiment, which was to determine the lutein content in mg lutein/1 g solids, it seems that the two soy based products (Soy Isoflavones and Pro Fam 892 Soy Isolated Protein), had the most lutein associated with them after the extractions. The results of the experiment showed that the soy based products, when mixed with lutein, had greater absorbance at 446 nm. This would lead one to believe that there is more lutein present in the presence of these proteins. Dilutions were made of all the samples before reading on UV/VIS to reduce background interference.

### EXAMPLE 11

Formulation of Prototype of Soy/Lutein Product in Juice Model

The purpose of this experiment was to determine if the lutein-protein products produced in our labs could be added to juices. Four grams protein samples (i.e., Chem West Soy Protein (lot#141193), ADM Soy Isoflavones Protein Product (lot#0011101), and Pro Fam Soy Protein Isolate S892 (lot#1032261)) were weighed. Each protein sample was dissolved in Tris buffer (400 ml) (Tris buffer=1.74 g Trizma preset crystals+0.42 g calcium chloride). Ethanol (10 ml) or lutein solution (40 mg in 50 ml of 95% ethanol), were added to each of the control and test samples, respectively.

After incubation, samples were placed in refrigerator (4° C.). Vials were then taken out, transferred to new vessels, centrifuged, and the supernatant discarded. All samples were extracted four times with methylene chloride (40 ml). Samples were placed on an analytical evaporator until excess solvent was removed, then they were lyophilized into powders over a 48-hour period.

In order to determine the binding efficiency, 20 mg of each sample was weighed and dissolved in 10 ml of a 20% sucrose ester solution. Samples were sonicated for fifteen minutes, and filtered through a 0.2 micron filter and absorbance at 446 nm was determined.

| | Estimated Lutein |
|---|---|
| | content (mg |
| Sample | lutein/1 g solids) |
| Solgen | 3.265 |
| CW Soy | 1.628 |
| ADM Soy | 6.587 |
| Isoflavone | |
| S892 | 2.555 |

The drink model was prepared as follows:

| Weight % | Ingredient |
|---|---|
| 72.71% | Water |
| 17.00% | High Fructose Corn Syrup |
| 10.00% | Juice |
| 00.25% | Citric Acid |
| 00.04% | Ascorbic Acid |
| 00.11% | Soy/Lutein Product¹ |

¹ Calculated from previous Isoflavone results of 0.4 mglutein/200 mg protein to yield approx. 0.5 mg lutein/beverage serving.

Six hundred grams of beverage were prepared according to this formulation and homogenized using a handheld 2 speed Bio-homogenizer for approximately two minutes. Samples of the beverage were aliquoted into 120 g glass jars and heat treated for 10 min in a boiling water bath (100° C.). Samples were left to cool on benchtop overnight and then samples were stored at 40° C. for 0, 4, 10, 14, 28 days. At each time point samples were removed from the 40° C. incubator and stored at −20° C. until analysis. Beverage prototype samples were analyzed as described previously using absorbance at 446 nm.

### EXAMPLE 12

Efficiency of Binding Lutein to Proteins Using Various Buffers

This experiment was designed to repeat the results of a previous experiment aimed at determining the effect of calcium chloride, on the binding of lutein to the Isoflavone Soy Protein product. Also, of interest is determining the binding efficiency when performed in glycine buffer.

A 0.2 M glycine buffer was prepared at pH 8.7 was prepared as follows: 250 ml glycine solution (7.505 g dissolved in 500 ml de-ionized water) were mixed with 2 ml NaOH (2 M) and the volume was brought to 1 liter with water. The Ca-, glycine buffer was prepared by dissolving 0.42 g of calcium chloride in 250 ml of the previously prepared glycine buffer. A carbonate/bicarbonate buffer (pH 8.9) was prepared by mixing 20 ml of solution A (6.2 g sodium carbonate monohydrate dissolved in 500 ml) with 180 ml of solution B (4.2 g sodium bicarbonate dissolved in 500 ml). The Ca-, carbonate/bicarbonate buffer solution (pH 7.9) was prepared similarly, however, 0.336 g of calcium chloride were added also. Finally, Tris buffer was prepared with (1.74 g Trizma preset crystals, 0.42 g calcium carbonate dissolved in 250 ml water, pH 8.4) and without calcium chloride (1.74 g Trizma preset crystals dissolved to 250 ml water, pH 8.5).

Twelve samples of Soy Isoflavones Protein Product (ADM lot#0011101) (200 mg) were weighed into twelve vessels. Twenty ml of each of the buffers was added to two of the protein samples. Ethanol (0.5 ml) or lutein solution (Dry cake, Kemin Foods, L.C.) (8 mg) were dissolved in 10 ml) were added to the control and test sample, respectively. All samples were incubated with vigorous mixing overnight.

After incubation, each sample was extracted with methylene chloride (3 times, 20 ml). Samples were then dried under reduced pressure on an analytical evaporator under nitrogen, followed by lyophilization for approximately five hours. Of the dried powder samples, 10 mg of each sample were taken. Total solids (without buffer salts) were determined by using a pre-weighed Microcon filter. Each sample was washed with de-ionized water (3 times, 0.2 ml), and dried under reduced pressure. Of each sample, another 10 mg aliquot was dissolved in sucrose ester solution (20%, 20 ml) and each sample was sonicated for 1 hr. Additional dilution (1:2) was performed on each sample (3 ml) using sucrose esters solution (20%) and each sample was sonicated for one hour. Absorbance at 446 nm was determined in each sample using a spectrophotometer.

| Sample | Exp. 11 | Exp. 13 |
|---|---|---|
| Bicarb w/o Ca | 0.48 | 0.42 |
| Bicarb w/Ca | 1.15 | 1.28 |
| Tris w/o Ca | 0.176 | 0.47 |
| Tris w/Ca | 0.195 | 0.47 |
| Glycine w/o Ca | 0.13 | 0 |
| Glycine w/Ca | | 0.49 |

The results of this experiment were similar to the results reported in the above table for the reaction in carbonate/bicarbonate buffer.

### EXAMPLE 13

Experiment Using Soy Isoflavone Protein Product and Five Different Carotenoids

The purpose of this experiment is to compare the performance of four other carotenoids (beta-carotene, lutein esters, zeaxanthin, and lycopene) to lutein using the previous experiments as a model.

The first step was to prepare the carbonate/bicarbonate buffer with calcium chloride. Six and two-tenths g of sodium carbonate monohydrate (lot#03313TV) diluted in 500 ml of de-ionized water, and 4.2 g of sodium bicarbonate (lot#016178A) diluted in 500 ml of de-ionized water was prepared. Then 50 ml of sodium bicarbonate and 450 ml of sodium carbonate monohydrate were mixed. To make the carbonate/bicarbonate buffer with calcium chloride, 0.168 g of calcium chloride was weighed into a 100 ml volumetric flask, and the previously mixed carbonate/bicarbonate mixture was poured into the flask until the volume reached 100 ml. Five carotenoid mixtures were prepared as follows: 4 mg of a carotenoid (lutein, beta-carotene, lutein esters, zeaxanthin, or lycopene) were weighed into a 7 ml vial, and 5 ml of THF was added. Two hundred milligrams of Soy Isoflavone Protein Product (ADM lot#0011101) were weighed into each of ten 20 ml scintillation vials. Twenty ml of the formulated buffer was added to each vial. One half of a milliliter of each carotenoid was added to the respective test vial, and 0.5 ml of THF was added to each control. All samples were incubated with vigorous mixing over night.

After incubation, each sample was extracted with methylene chloride (3 times, 20 ml). Samples were then dried under reduced pressure on an analytical evaporator under nitrogen, followed by lyophilization for approximately five hours. Of the dried powder samples, 10 mg of each sample were taken. A determination of total solids (without buffer salts) was performed using a pre-weighed Microcon filter. Each sample was washed with de-ionized water (3 times, 0.2 ml), and dried under reduced pressure. Another 10 mg aliquot was dissolved in sucrose ester solution (20%, 20 ml) and each sample was sonicated for 1 hr. Absorbance at 445 nm, 446 nm, 450 nm, and 470 nm was determined in each respective sample using a spectrophotometer.

| | Estimated Carotenoid Content |
|---|---|
| Sample | (mg carotenoid/1 g protein) |
| Beta carotene test | 0 |
| Lutein esters test | 0 |
| Lycopene test | 0.0647 |
| Zeaxanthin test | 1.200 |
| Lutein | 0.617 |

While the foregoing examples have included processes that resulted in a distinct protein and carotenoid product that is used to supplement a finished food, beverage, or personal care product, it is logical to expect this protein and carotenoid complex to form in foods, beverages, personal care products or dietary supplements in which the right conditions (e.g., proteins and minerals such as calcium or iron, the right protein:carotenoid ratio and temperature) required for the reaction to take place could prevail, upon the inclusion of lutein or other carotenoids,. Many of the currently available foods and beverages naturally contain proteins and minerals or are fortified with added proteins and minerals in order to achieve nutritional benefits. Examples of such foods and beverages include, but are not limited to soymilk, baby formulas and other soy products, meal-replacement beverages, weight loss beverages, high protein drinks, ready-to-eat cereals, hot cereals, cereal and nutritional bars, and dairy products. These foods and beverages undergo a variety of processing conditions, examples of which include homogenization, high shear mixing, pasteurization, extrusion, and retort. Additionally, the production of these foods and beverages involve the formation of solutions, suspensions, slurries, pastes or other mixtures. The presence of proteins and minerals with the addition of lutein in these products, combined with the aforementioned processing conditions provide similar conditions in foods and beverages for the formation of the lutein-protein complex described in this invention. Similarly, this complex will form as part of the production process, in liquid supplements, gel caps, nutritional bars, beverages and beverage powders, confectionary candies, and other dietary supplement forms.

Although the invention has been described with respect to a preferred embodiment thereof, it is to be also understood that it is not to be so limited since changes and modifications can be made therein which are within the full intended scope of this invention as defined by the appended claims.

We claim:

1. A method for forming a protein and carotenoid product, comprising the steps of:
(a) preparing a solution of a carotenoid in a solvent;
(b) adding a source of metal ions;
(c) adding a source of protein; and
(d) incubating the solution.

2. A method as defined in claim 1, wherein the carotenoid comprises esters of hydroxyl- or carboxyl-containing carotenoid.

3. A method as defined in claim 1, wherein the carotenoid is selected from the group comprising actinioerythrol, astaxanthin, bixin, canthaxanthin, capsanthin, capsorubin, -8′-apo-carotenal (apo-carotenal), -12′-apo-carotenal, -carotene, -carotene, “carotene” (a mixture of - and -carotenes), -carotene, -cryptoxanthin, lutein, lycopene, violerythrin, zeaxanthin, and esters of hydroxyl- or carboxyl-containing members thereof.

4. A method as defined in claim 1, wherein the carotenoid comprises xanthophylls and esters of hydroxyl- or carboxyl-containing members thereof.

5. A method as defined in claim 1, wherein the metal ions are selected from the group comprising Mg⁺⁺, Mn⁺⁺, Fe⁺⁺⁺, K⁺, and Ca⁺⁺.

6. A method as defined in claim 1, wherein the solvent is selected from the group comprising tetrahydrofuran, methanol, ethanol, propylene glycol, methylene chloride, and isopropyl alcohol.

7. A method as defined in claim 1, wherein the protein source is selected from the group comprising soybean proteins and isolates; albumin from various sources (i.e., bovine and chicken); whey proteins, concentrates and isolates; isoflavone containing protein products; corn germ proteins; oat flour; soybean proteinase inhibitors, comprising Kunitz, Bowman-Birk, and carboxypeptidase inhibitors; potato proteinase inhibitors and others; and hydrolyzed vegetable proteins.

8. A method as defined in claim 1, further comprising the step of drying to form a dry product containing the protein and carotenoid product.

9. A method for forming a protein and carotenoid product, comprising the steps of:
(a) adding a carotenoid to a protein-containing mixture that also contains a source of metal ions; and
(b) incubating and heating the mixture.

10. A composition of a carotenoid and a protein, comprising a product formed by the method of claim 1.

11. A personal care composition, comprising a carotenoid and protein product of claim 10 added to a personal care product.

12. A beverage composition, comprising a carotenoid and protein product of claim 10 added to a beverage.

13. A food composition, comprising a carotenoid and protein product of claim 10 added to a food.

14. A composition of a carotenoid and a protein for addition to a food, beverage, or personal care product, comprising a product formed by the method of claim 1.

15. A method of supplementing a food, beverage, dietary supplement, or personal care product with a protein, a carotenoid, or both, comprising the step of adding to a food, beverage, or personal care product a carotenoid and protein product formed by the method of claim 1.
